(19) United States
(12) Patent Application Publication
(10) Pub. No.: US 2015/0230754 A1
(43) Pub. Date: Aug. 20, 2015

(54) Restraint System

(71) Applicant: Kevin Willis, San Jose, CA (US)

(72) Inventor: Kevin Willis, San Jose, CA (US)

(21) Appl. No.: 14/622,036

(22) Filed: Feb. 13, 2015

Related U.S. Application Data
(60) Provisional application No. 61/939,842, filed on Feb. 14, 2014.

(51) Int. Cl. A61B 5/00; A61F 5/37; A61B 5/024

(52) U.S. Cl. CPC A61B 5/6831; A61B 5/02433; A61B 5/7445; A61B 5/02438; A61F 5/3723; A61B 2560/0214; A61B 2562/164

(57) ABSTRACT

A restraint system for restraining a user and monitoring the pulse of the user at the time he or she is administered cardiopulmonary resuscitation (CPR). The restraint system prevents the user from harming himself or medical professionals during an emergency situation. The restraint system includes a pair of cuffs connected by a strap, wherein each cuff includes a means for monitoring a user's pulse, such as an electrode, infrared sensor, or other similar device. The cuffs can be secured on the user's wrists, wherein the strap is used to restrain excess arm movement of the user. A display is disposed on the strap, wherein the display is electrically connected to the sensors. The display shows the pulse rate of the user, thereby providing assistance to an emergency medical professional in assessing the user's health status and aiding in the administration of the correct compression rates during CPR.

### CROSS REFERENCE TO RELATED APPLICATION

This application claims the benefit of U.S. Provisional Application No. 61/939,842 filed on Feb. 14, 2014. The above identified patent application is herein incorporated by reference in its entirety to provide continuity of disclosure.

### BACKGROUND OF THE INVENTION

### Field of the Invention

The present invention relates to a restraint system. More specifically, the present invention provides a restraint system comprising a strap having a first side opposite a second side, wherein a first cuff is attached to the first side of the strap and a second cuff is attached to the second side of the strap. The cuffs can be removably secured on a user's wrists, wherein the restraint system is adapted to restrain excess arm movement of the user. Each cuff includes means for determining the user's pulse rate or other physiological information, such as one or more infrared sensors, electrodes, or other similar devices. The strap further includes a display thereon, wherein the display is adapted to show physiological information, such as the pulse rate as determined by the infrared sensors or electrodes.

First responders and emergency medical professionals commonly perform cardiopulmonary resuscitation (CPR). Patients who receive CPR and other advanced life support operations have a greater chance of survival. When CPR is initiated, a patient is often placed on the floor in order to provide a solid support structure underneath the patient. After CPR is performed, the patient must be transported to a hospital or similar facility in an ambulance gurney. While loading a patient onto an ambulance gurney, it can be difficult to control the patient's upper extremities. The arms of a patient may dangle limply over the sides of the gurney or may flail about. This type of body movement can result in trauma and injury to the patient. If the upper extremities of a patient are not controlled, the extremities can be scratched, bruised, pinched, or even crushed by the mechanical components on the floor of the ambulance. Family members and friends that are present at the scene may become distraught at the sight and may develop the impression that proper procedures are not being administered in a controlled and professional manner. Furthermore, when performing CPR, it is important to monitor a patient's pulse rate in order to administer the correct compression rates. A numerical pulse rate display can assist in the inference of systolic blood pressure by viewing the radial pulse rate.

Devices have been disclosed in the prior art that relate to sensory devices. These include devices that have been patented and published in patent application publications. These devices generally relate to textiles or bracelets that contain an electrical conductor used to detect physiological information of the wearer. Such devices include U.S. Pat. No. 5,471,983 to Magnus, U.S. Pat. No. 8,295,919 to Edinburgh, U.S. Pat. No. 7,878,030 to Burr, U.S. Publication Number 2007/0023496 to Hall, U.S. Publication Number 2011/0275915 to Allgeyer, and U.S. Publication Number 2012/0283583 to Batkin et al.

These patents and patent publications generally disclose devices used to monitor a person's location or to monitor functions inside a human body, such as heart irregularities, by detecting electrical activity with a sensor. The sensor may be located in a garment, such as a shirt, or a single bracelet worn around an arm. These devices, however, fail to provide a restraint system comprising a single strap connecting a first cuff and a second cuff, therefore providing a means to restrain a user for his or her protection during an emergency situation. Furthermore, these devices fail to provide a restraint system further comprising a digital display, wherein the display is adapted to show the user's pulse rate, thereby providing assistance to an emergency medical professional in assessing the user's health status and aiding in the administration of the correct compression rates during CPR.

In light of the devices disclosed in the prior art, it is submitted that the present invention substantially diverges in design elements from the prior art and consequently it is clear that there is a need in the art for an improvement to existing restraint devices. In this regard the instant invention substantially fulfills these needs.

### SUMMARY OF THE INVENTION

In view of the foregoing disadvantages inherent in the devices known in the prior art, the present invention provides a new restraint system wherein the same can be utilized for providing protection for the user while the user is being transported or while the user is receiving emergency medical treatment.

It is therefore an object of the present invention to provide a new and improved restraint system that has all of the advantages of the prior art and none of the disadvantages.

It is another object of the present invention to provide a restraint system comprising a strap having a first side opposite a second side and an upper surface, wherein a first cuff is attached to the first side of the strap and a second cuff is attached to the second side of the strap, wherein the device further includes a display disposed on the upper surface of the strap.

Another object of the present invention is to provide a restraint system wherein the first cuff and second cuff are padded and the strap is composed of fabric so to provide protection to the user while restrained.

Yet another object of the present invention is to provide a restraint system having a means for monitoring the user's pulse, such as one or more infrared sensors, electrodes, or the like, wherein the means for monitoring a user's pulse are disposed on the cuffs and are operably connected to the display, such that the display is adapted to show the pulse rate of the user.

Yet another object of the present invention is to provide a restraint system having a power source adapted to provide power to the display and infrared sensors.

Yet another object of the present invention is to provide a restraint system wherein the first cuff and the second cuff comprise a loop of material adapted to be wrapped around a user's wrist and secured in an overlapping relation by fasteners on opposing ends of the cuff.

Another object of the present invention is to provide a restraint system that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the restraint system.

FIG. 2A shows a top down view of a single cuff in an open configuration.

FIG. 2B shows a perspective view of a single cuff in a closed configuration.

FIG. 3 shows a diagram of the control circuit of the restraint system.

FIG. 4 shows a perspective view of the restraint system in use.

### DETAILED DESCRIPTION OF THE INVENTION

Reference is made herein to the attached drawings. Like reference numerals are used throughout the drawings to depict like or similar elements of the restraint system. For the purposes of presenting a brief and clear description of the present invention, the preferred embodiment will be discussed as used for restraining a user and monitoring the pulse of a user while he or she receives CPR and is subsequently loaded onto a gurney. The figures are intended for representative purposes only and should not be considered to be limiting in any respect.

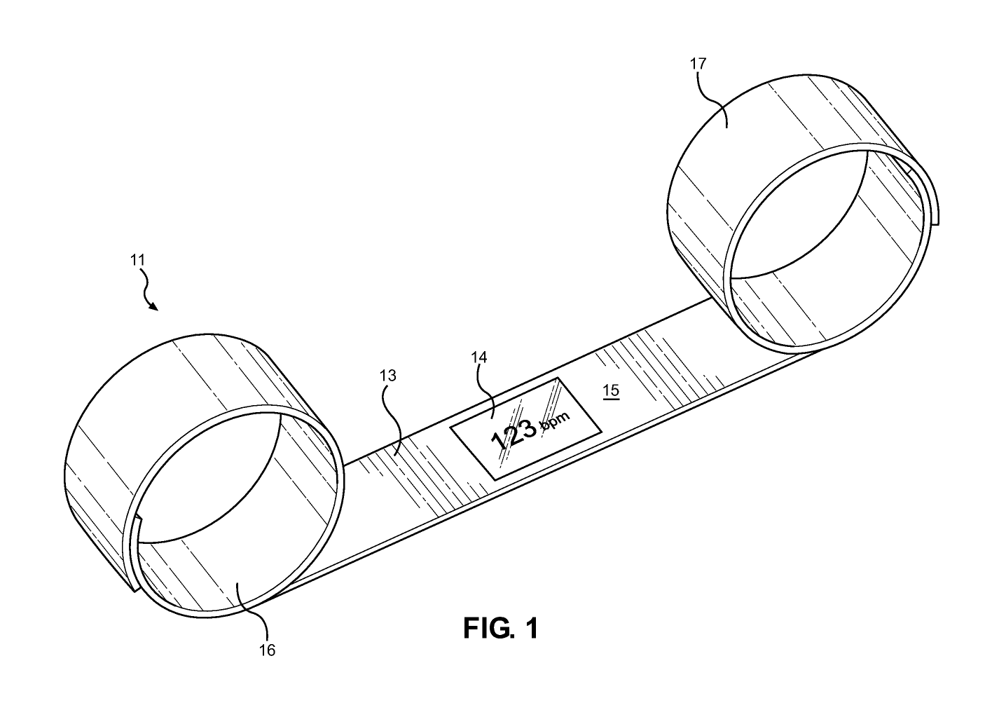

Referring now to FIG. 1, there is shown a perspective view of an embodiment of the restraint system 11 of the present invention. The restraint system 11 comprises a strap 13 having a first side opposite a second side and an upper surface 15, wherein a first cuff 16 is attached to the first side of the strap 13 and a second cuff 17 is attached to the second side of the strap 13. The first cuff 16 and second cuff 17 are permanently connected to the strap 13 by any suitable fastener, such as adhesive or stitching. The first cuff 16 and the second cuff 17 are preferably identical in structure.

The strap 13 is elongated, substantially flat and rectangular in configuration. The strap 13 is flexible and can be made from cloth or synthetic polymer. The length of the strap 13 is similar to the width of a user's torso so when the user is wearing the cuffs around his or her wrists, his or her arms are restrained to the side of his or her body. In some embodiments, the strap 13 is adjustable in length and includes an adjustment mechanism, such as a buckle, for allowing the user or emergency medical professional to selectively elongate the strap 13 depending upon the size of the user. A digital display 14 is permanently affixed on the upper surface 15 of the strap 13 by any suitable fastener, such as adhesive. The display 14 is disposed centrally on the strap 13. The display 14 is preferably substantially rectangular or square in configuration, but may be provided in alternate configurations.

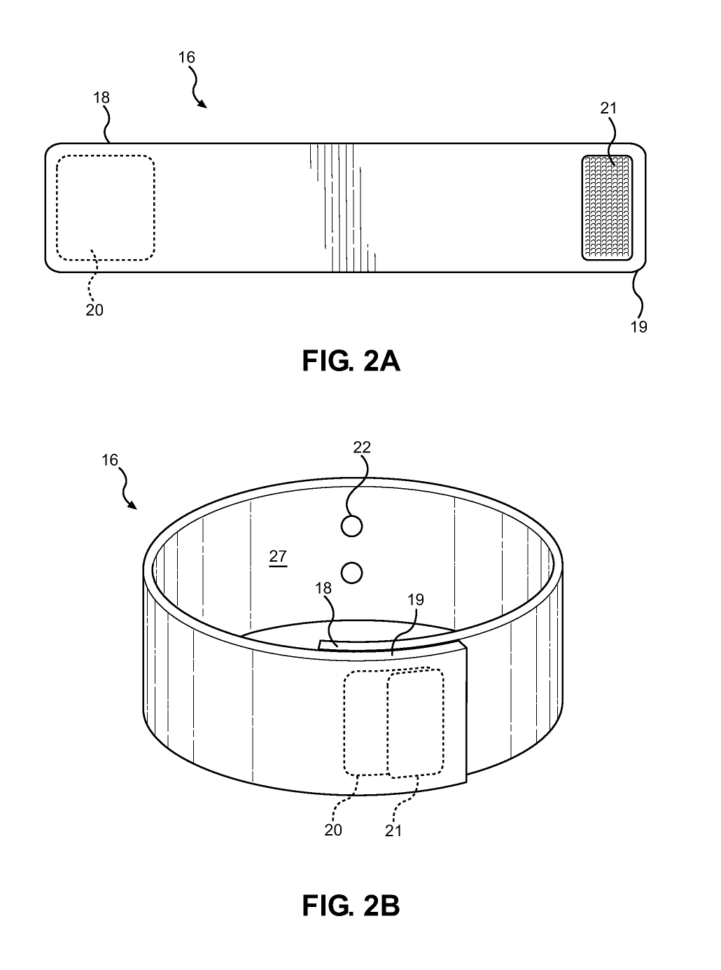

Referring now to FIGS. 2A and 2B, there is shown a top down view of the first cuff 16 in an open configuration and a perspective view of the first cuff 16 in a closed configuration, respectively. The cuff 16 comprises an outer surface, an inner surface 27, and a first end 18 opposite a second end 19. Preferably, hook and loop fastening material 20 is disposed on the outer surface of the first end 18 of the cuff 16 and mating hook and loop fastening material 21 is disposed on the inner surface 27 of the second end 19 of the cuff 16. However, in alternate embodiments, other types of fasteners may be used. As illustrated in FIG. 2B, the cuff 16 can be secured in a closed configuration by folding the second end 19 of the cuff 16 over the first end 18 thereof, wherein the fastening material 20, 21 thereon engages in an overlapped configuration, thereby fastening the first end 18 of the cuff with the second end 19 of the cuff. The cuff 16 is composed of a flexible material adapted to be wrapped around a user's wrist. A circular configuration is formed when the cuff 16 is in a closed configuration. The cuff 16 may be composed of cloth or a synthetic polymer, among other suitable materials. In some embodiments, the cuff 16 includes padded material so to provide additional comfort to the user when wearing the cuff 16 around his or her wrist. The width of the cuff 16 is substantially similar to the width of the strap. When in a closed configuration, the hook and loop material may be overlapped at different points, thereby permitting the circumference of the cuff 16 to vary depending on the size of the user's wrist.

The restrain system provides a means for detecting a wearer's pulse, wherein the means for detecting a wearer's pulse can include the use of one or more infrared sensors, electrodes, or other suitable devices. Preferably, each cuff of the restraint system includes one or more infrared sensors 22 adapted to measure the user's physiological information, such as pulse rate. The infrared sensors 22 are preferably circular in configuration and are disposed on the inner surface 27 of the cuff 16, such that the infrared sensors 22 can be in contact with the inner surface of the user's wrist when the cuff 16 is secured thereon. In other embodiments, the infrared sensors 22 can be disposed within the interior of the cuff 16 so the infrared sensor 22 is not exposed. The infrared sensors 22 are in electrical communication with a display so as to inform the user or a healthcare professional as to the wearer's pulse rate and/or other physiological information.

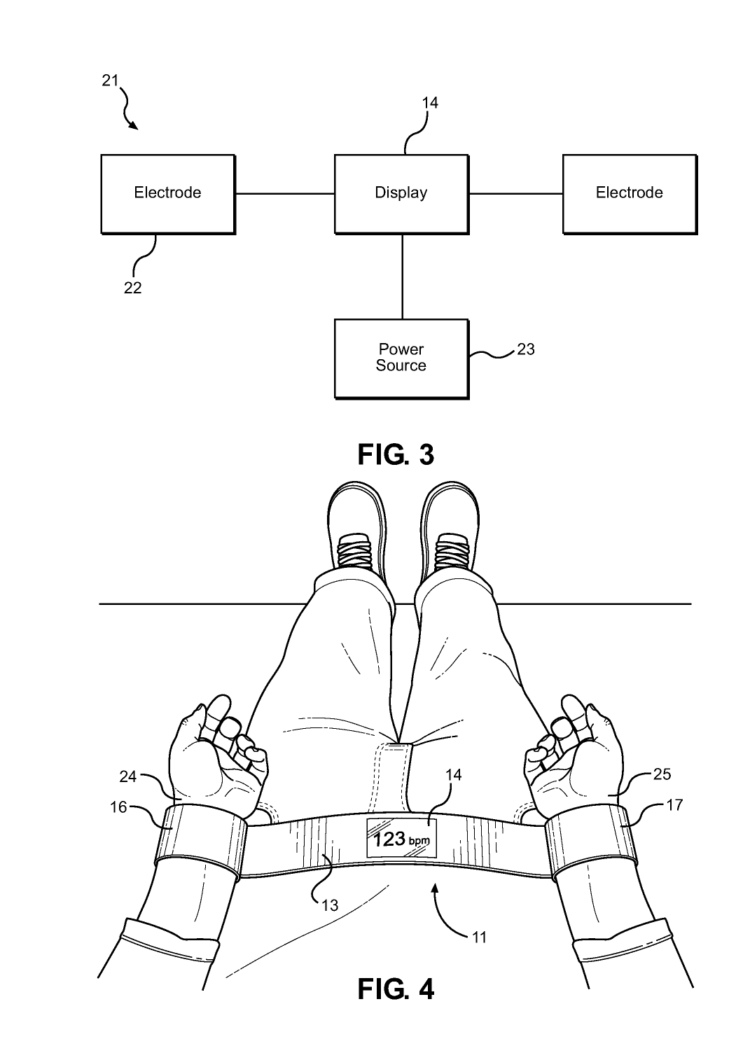

Referring now to FIG. 3, there is shown a diagram of the control circuit of the restraint system. The restraint system includes a control circuit 21 in which the means for monitoring a user's pulse, such as infrared sensors, are in communication with the display 14. The infrared sensors 22 in the cuff measure and monitor physiological information, such as the pulse rate of the wearer, and communicate the information to a display 14. The display 14 indicates to the user the user's pulse rate and/or other physiological information. A power source 23 powers the display 14 and is electrically connected thereto. The power source 23 may be one or more disposable or rechargeable batteries, wherein the batteries can be lithium ion, alkaline, or the like.

Referring now to FIG. 4, there is shown a perspective view of the restraint system 11 in use. In operation, an emergency medical professional can wrap the open, elongated first cuff 16 around the user's left wrist 24. The first end of the cuff 16 can be overlapped with the second end of the cuff, and the fasteners thereon can be secured in an overlapping configuration, whereby the cuff 16 is fastened securely in a closed, circular configuration around the user's wrist 24. The second cuff 17 can be secured around the user's right wrist 25 in a similar fashion. Infrared sensors positioned on each cuff 16, 17 are in contact with the user so as to measure or monitor the user's pulse rate or other physiological information. When both cuffs are secured on the user's wrist, he or she is restrained, and is therefore unable to erratically move his or her arms while the medical professional is administering CPR or loading the user onto a gurney. The strap 13 connecting the pair of cuffs 16, 17 rests across the user's torso.

The display 14 located on the strap 13 displays physiological information about the user, such as the user's pulse rate, to help emergency personnel monitor the wearer's vitals and to help the emergency personnel to administer CPR. The display 14 may include one or more control switches for turning the display 14 on or off and for controlling the operation of the display 14. A power source provides the necessary power to illuminate the display 14. From the numerical pulse rate viewable on the display 14, the medical professional is able to administer correct compression rates during CPR.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A restraint system, comprising:
a strap having a first side opposite a second side and an upper surface;
a first cuff attached to said first side of said strap and a second cuff attached to said second side of said strap;
a display disposed on said upper surface of said strap, wherein said display is adapted to show digital physiological measurements.

2. The restraint system of claim 1, wherein said first cuff and said second cuff comprise padding thereon.

3. The restraint system of claim 1, wherein said strap is composed of fabric.

4. The restraint system of claim 1, wherein said first cuff and said second cuff each include one or more infrared sensors adapted to monitor a user's pulse rate.

5. The restraint system of claim 4, wherein said one or more infrared sensors are operably connected to said display such that said display is adapted to show the pulse rate of the user.

6. The restraint system of claim 1, wherein said display is disposed on the center of said upper surface of said strap.

7. The restraint system of claim 1, wherein said strap comprises a power source.

8. The restraint system of claim 7, wherein said power source comprises one or more batteries.

9. The restraint system of claim 1, wherein said first cuff and said second cuff comprise a loop of material adapted to be wrapped around a user's wrist.

10. The restraint system of claim 1, wherein said first cuff and said second cuff comprise fasteners on opposing ends thereof that can be secured in overlapping relation.

11. The restraint system of claim 10, wherein said fastener comprises hook and loop fastening material.
